(19) United States
(12) Patent Application Publication
(10) Pub. No.: US 2025/0152411 A1
(43) Pub. Date: May 15, 2025

(54) CORE BODY TEMPERATURE MEASUREMENT

(71) Applicant: Masimo Corporation, Irvine, CA (US)

(72) Inventors: Valery G. Telfort, Irvine, CA (US); Philip Perea, Irvine, CA (US); Jerome Novak, Lake Forest, CA (US)

(21) Appl. No.: 19/025,501

(22) Filed: Jan. 16, 2025

Related U.S. Application Data
(63) Continuation of application No. 18/529,403, filed on Dec. 5, 2023, now Pat. No. 12,257,183.
Application No. 18/529,403 is a continuation of application No. 16/546,667, filed on Aug. 21, 2019, now Pat. No. 11,872,156.
(60) Provisional application No. 62/721,062, filed on Aug. 22, 2018.
(60) Provisional application No. 62/810,491, filed on Feb. 26, 2019.

(51) Int. Cl. A61F 7/02; A61B 5/01; A61B 5/026; A61F 7/00

(52) U.S. Cl. CPC A61F 7/02; A61B 5/01; A61B 5/026; A61B 2560/0252; A61B 2562/0271; A61F 2007/0001; A61F 2007/0075; A61F 2007/0093; A61F 2007/0096; A61F 2007/0288

(57) ABSTRACT

Continuous core body temperature measurements are made during hypothermic operations, where the core body temperature of the patient is lowered to reduce swelling. Caregivers monitor the patient's core body temperature to prevent damage that can occur to the patient if the patient's core body temperature becomes too low. To accurately determine the core body temperature of the patient, a temperature monitoring system measures the temperature at or near the surface of the patient and through at least a portion of a thermal block at multiple locations.

### CROSS REFERENCE TO RELATED APPLICATIONS

This application is a continuation of U.S. patent application Ser. No. 18/529,403, filed Dec. 5, 2023, which is a continuation of U.S. patent application Ser. No. 16/546,667, filed Aug. 21, 2019, which claims priority to U.S. Provisional Patent Application No. 62/721,062, filed Aug. 22, 2018 and to U.S. Provisional Patent Application No. 62/810,491 filed Feb. 26, 2019, which are incorporated herein by reference in their entireties. Any and all applications, if any, for which a foreign or domestic priority claim is identified in the Application Data Sheet of the present application are hereby incorporated by reference under 37 CFR 1.57.

### FIELD

The present disclosure relates generally to the field of measuring core body temperature, and in particular, to measuring core body temperature at multiple body sites to increase accuracy.

### BACKGROUND

During hypothermic operations, the core body temperature of the patient is lowered to a predetermined level. Lowering the patient's core body temperature can reduce swelling of the patient during surgery and can expedite the patient's recovery time after the surgery. As the core body temperature of the patient decreases, the patient's heart rate, respiratory rate, blood pressure, and metabolic processes decrease. If the core body temperature of the patient becomes too low, the patient's cellular metabolic processes can shut down, which can lead to organ failure or death. To avoid the deleterious effects of unintentional over-cooling, the core body temperature of the patient may be constantly monitored.

### SUMMARY

There is a need to provide continuous and accurate core body temperature measurements of a patient during a medical procedure during which core body temperature is intentionally reduced.

Aspects of the present disclosure are directed to accurately monitoring core body temperature. Embodiments generally include a temperature monitoring device that includes a thermal block having known dimensions and known thermal conductivity, and a first temperature measuring device coupled to the thermal block. The first temperature measuring device may be coupled to a surface of the thermal block or may be located within the thermal block. The temperature monitoring device can further include a second temperature measuring device. The second temperature measuring device may be coupled to a surface of the thermal block or may be located within the thermal block. The first and second temperature measuring devices may be located on opposing sides of the thermal block. The first and second temperature measuring devices can be located a known distance from each other on or within the thermal block.

A temperature monitoring system can include two temperature monitoring devices. The two temperature monitoring devices can be disposed at different locations on or near a patient's skin. Each temperature monitoring device can include a thermal block having known dimensions and known thermal conductivity and at least one temperature measuring device that is coupled to a surface of the thermal block or is located within the thermal block. Each temperature monitoring device can include a thermal block having known dimensions and known thermal conductivity and two temperature measuring devices. The two temperature measuring devices for each thermal block may be coupled to opposing sides of the thermal block, located within the thermal block a known distance apart, or one temperature measuring device may be located on a surface of the thermal block and the other temperature measuring device may be located within the thermal block. Each thermal block can have the same or different thermal conductivity. The temperature measuring devices can be temperature sensors, such as thermistors, resistance temperature detectors, thermocouples, semiconductor-based temperature sensors, and the like.

The temperature monitoring system can use the temperature measured by each temperature measuring device and the known thermal conductivities and dimensions of the thermal block(s) to determine the patient's core body temperature.

Temperature monitoring systems that use a single temperature monitoring device make assumptions about the thermal conductivity of the patient's skin to determine the patient's core body temperature. Assumptions about the thermal conductivity of the patient's skin can lead to errors when determining the patient's core body temperature.

Temperature monitoring systems that use at least two temperature monitoring devices can determine the patient's core body temperature without making assumptions about the thermal conductivity of the patient's skin. Because assumptions about the thermal conductivity of the patient's skin do not need to be made when at least two temperature monitoring devices are used, the temperature monitoring system having at least two temperature monitoring devices is more accurate than the temperature monitoring system having one temperature monitoring device. In addition, the temperature monitoring system that includes at least two temperature monitoring devices can also determine air temperature, such as the ambient air temperature.

Aspects of this disclosure relate to a temperature monitoring system comprising a first temperature monitoring device configured to be disposed at first location associated with a patient, where the first temperature monitoring device comprises a first temperature measuring device, a second temperature measuring device, and a first thermal block having a first thermal conductivity. At least a portion of the first thermal block can be between the first and second temperature measuring devices. The first temperature measuring device can be configured to measure a first temperature associated with the patient at the first location, and the second temperature measuring device can be configured to measure a second temperature through the at least a portion of the first thermal block.

The temperature monitoring system can further comprise a second temperature monitoring device configured to be disposed at a second location associated with the patient, where the second temperature monitoring device can comprise a third temperature measuring device, a fourth temperature measuring device, and a second thermal block having a second thermal conductivity. At least a portion of the second thermal block can be between the third and fourth temperature measuring devices. The third temperature measuring device can be configured to measure a third temperature associated with the patient at the second location, and the fourth temperature measuring device can be configured to measure a fourth temperature through the at least a portion of the second thermal block.

The temperature monitoring system can further comprise one or more processors configured to receive temperature measurements from the first, second, third, and fourth temperature measuring devices, and based on the received temperature measurements, the first and second thermal conductivities, a first distance between the first and second temperature measuring devices, and a second distance between the third and fourth temperature measuring devices, can determine a core body temperature of the patient.

The one or more processors can be further configured to determine a skin thermal resistance of the patient. The one or more processors can be further configured to determine an ambient air temperature. The first temperature measuring device can be further configured to measure the first temperature at or near a surface of the patient at the first location and the third temperature measuring device can be further configured to measure the third temperature at or near the surface of the patient at the second location. A first side of the first thermal block can be over the first location and a first side of the second thermal block can be over the second location. The second temperature measuring device can be configured to measure the second temperature at a second side opposite the first side of the first thermal block and the fourth temperature measuring device can be configured to measure the fourth temperature at a second side opposite the first side of the second thermal block. At least one of the first, second, third, and fourth temperature measuring devices can include a thermistor.

The temperature monitoring system can further comprise a display configured to display the core body temperature of the patient. The first and second distances can be approximately the same. The first and second thermal conductivities can be approximately the same. The first and second distances can also be different. The first and second thermal conductivities can also be different.

Aspects of this disclosure relate to a temperature monitoring system comprising at least two passive temperature monitoring devices configured to be disposed at respective locations associated with a patient. Each passive temperature monitoring device can comprise a thermal block between at least portions of first and second temperature measuring devices. Each first temperature measuring device can be configured to measure a temperature at the respective location. Each second temperature measurement device can be configured to measure a temperature through at least a portion of the corresponding thermal block. The temperature monitoring system can further comprise one or more processors configured to determine a core body temperature of the patient responsive to the temperature measurements and physical properties associated with the thermal blocks.

The physical properties associated with the thermal blocks can include a thermal conductivity and a thickness. For each passive temperature monitoring device, the thickness of the thermal block can represent a distance between the first and second temperature measuring devices.

Aspects of this disclosure relate to a temperature monitoring system comprising first and second temperature measuring devices, where the first temperature measuring device can be configured to be disposed at a first location associated with a patient; an insulator having a known thermal conductivity, where at least a portion of the insulator can be disposed between the first and second temperature measuring devices; and a Peltier device can be disposed over the second temperature measuring device. The temperature monitoring system can further comprise one or more processors configured to cool the insulator to a first temperature using the Peltier device; disable the Peltier device once the first temperature is reached; receive temperature measurements from the first and second temperature measuring device after disabling the Peltier device; determine a recovery time for the received temperature measurement from the first temperature measurement device to become approximately equal to the received temperature measurement from the second temperature measurement device; and determine perfusion information based at least in part on the recovery time.

The one or more processors can be further configured to determine patient care instructions based at least in part on the perfusion information. The first temperature can be approximately 2 degrees less than a temperature of the patient's skin at the first location. The first temperature can be approximately 5 degrees less than a temperature of the patient's skin at the first location. The recovery time can be based at least in part on heat flow between the first and second temperature measurement devices through the insulator.

For purposes of summarizing the disclosure, certain aspects, advantages and novel features of the innovations have been described herein. It is to be understood that not necessarily all such advantages may be achieved in accordance with any particular embodiment. Thus, the innovations may be embodied or carried out in a manner that achieves or optimizes one advantage or group of advantages as taught herein without necessarily achieving other advantages as may be taught or suggested herein.

### BRIEF DESCRIPTION OF THE DRAWINGS

Various embodiments will be described hereinafter with reference to the accompanying drawings. The drawings and the associated descriptions are provided to illustrate embodiments of the present disclosure and do not limit the scope of the claims. In the drawings, similar elements have similar reference numerals.

FIG. 1 is a block diagram of an example of a multi-site temperature monitoring system.

FIG. 1A is a block diagram illustrating an example of a single-site passive heat flux measurement system.

FIG. 2 is a block diagram of an example of a multi-site temperature monitoring system.

FIG. 3 is a circuit block diagram representation of an example a multi-site temperature monitoring system.

FIG. 4 is a circuit representation of an example multi-site temperature monitoring system.

FIG. 5 is a flow diagram illustrating an example process to accurately measure core body temperature using a multi-site temperature monitoring system.

FIG. 6A is a block diagram of an example of an active heat flux measurement system.

FIG. 6B is a block diagram of an example of another active heat flux measurement system.

FIG. 6C is a flow chart of an example process to obtain perfusion information of a patient using a Peltier active heat flux measurement system of FIG. 6B.

### DETAILED DESCRIPTION

Although certain embodiments and examples are described below, this disclosure extends beyond the specifically disclosed embodiments and/or uses and obvious modifications and equivalents thereof. Thus, it is intended that the scope of this disclosure should not be limited by any particular embodiments described below.

Overview of Core Temperature Measurement from Multiple Sites System

Aspects of the present disclosure describe a temperature monitoring system that measures temperature at or within thermal blocks that are disposed at multiple locations associated with a patient to determine the patient's core body temperature.

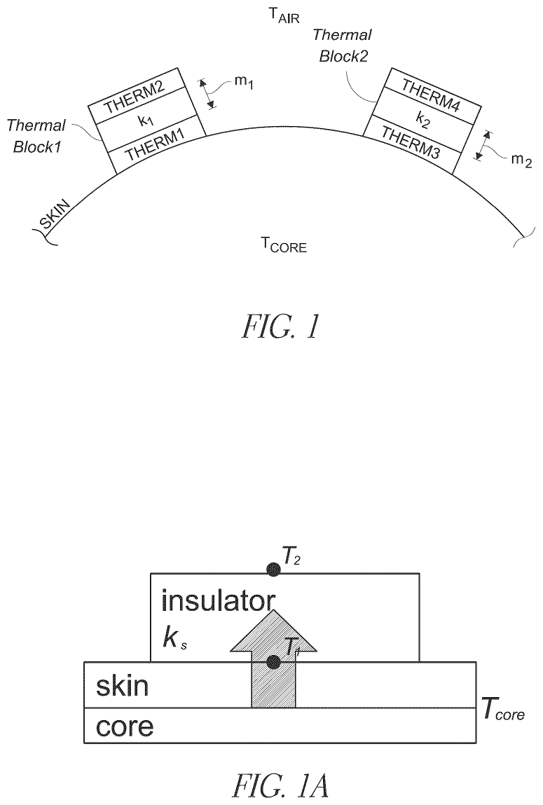

FIG. 1A is a block diagram of an example passive heat flux measurement system. In the illustrated passive heat measurement system of FIG. 1A, thermistor T1 can be positioned on top of the skin, an insulator or thermal block with a known thermal conductivity kₛ can be over thermistor T1, and thermistor T2 can be over the insulator. Heat flux from the skin at thermistor T1 to thermistor T2 can be measured. The temperature can calculated from the measured heat flux and the core body temperature is related to the calculated temperature. The core body temperature can be inferred from the calculated temperature. Using a single passive heat flux measurement system to infer the core body temperature based on the measured heat flux may use an estimate of the skin resistance R_{SKIN}. Using an estimate of the skin resistance R_{SKIN} can result in an inferred core body temperature that is inaccurate or less accurate because of the estimated the skin resistance R_{SKIN}.

Greater accuracy for the core body temperature can be achieved by using a plurality of passive heat flux measurement systems at multiple locations on the skin of the patient. Using at least two passive heat flux measurement systems and solving at least two equations with two unknown variables, as described herein, can provide greater accuracy in the core body temperature measurement. The two unknown variables can be core body temperature and skin resistance. By using the temperature measurements from at least two passive heat flux measurement systems and the physical properties of the passive heat flux measurement systems, the core body temperature can be determined with greater accuracy because the skin resistance does not need to be estimated.

FIG. 1 is a block diagram of an example multi-site temperature monitoring system. The temperature monitoring system can comprise multiple passive heat flux measurement systems. The temperature monitoring system can include a first temperature monitoring device that can include a first thermal block (Thermal Block 1), a first temperature measuring device (THERM 1), and a second temperature measuring device (THERM 2). The temperature monitoring system can further include a second temperature monitoring device that can include a second thermal block (Thermal Block 2), a third temperature measuring device (THERM 3), and a fourth temperature measuring device (THERM 4). The patient's skin is indicated as SKIN; the patient's core body temperature is indicated as T_{CORE}; and the air temperature is indicated as T_{AIR}.

The first temperature monitoring device comprising the first temperature measuring device THERM 1, the first thermal block Thermal Block 1, and the second temperature measuring device THERM 1 can be placed at a first location associated with the surface of the patient.

For example, the first temperature measuring device THERM 1 can be placed on the patient's skin, on a covering that is on the patient's skin, or near the patient's skin at the first location associated with the patient. The first thermal block Thermal Block 1 can be placed over the first temperature measuring device THERM 1. The first temperature measuring device THERM 1 can be located within the first thermal block Thermal Block 1 at the first location. The first temperature measuring device THERM 1 can be placed on an outer surface of the first thermal block Thermal Block 1.

The second temperature measuring device THERM 2 can be placed over the first thermal block Thermal Block 1. The second temperature measuring device THERM 2 can be located within the first thermal block Thermal Block 1 at the first location and separated from the first temperature measuring device THERM 1 by a known distance. The second temperature measuring device THERM 2 can be located on an outer surface of the first thermal block Thermal Block 1. The outer surface of the first thermal block Thermal Block 1 associated with the second temperature measuring device THERM 2 may be opposed to an outer surface of the first thermal block Thermal Block 1 associated with the first temperature measuring device THERM 1.

The second temperature monitoring device comprising the third temperature measuring device THERM 3, the second thermal block Thermal Block 2, and the fourth temperature measuring device THERM 4 can be placed at a second location associated with the surface of the patient.

For example, the third temperature measuring device THERM 3 can be placed on the patient's skin, on a covering that is on the patient's skin, or near the patient's skin at a second location, different from the first location, and associated with the patient. The second thermal block Thermal Block 2 can be placed over the third temperature measuring device THERM 3. The third temperature measuring device THERM 3 can be located within the second thermal block Thermal Block 2 at the second location. The third temperature measuring device THERM 3 can be placed on an outer surface of the second thermal block Thermal Block 2.

The fourth temperature measuring device THERM 4 can be placed over the second thermal block Thermal Block 2. The fourth temperature measuring device THERM 4 can be located within the second thermal block Thermal Block 2 at the second location and separated from the third temperature measuring device THERM 3 by a known distance. The fourth temperature measuring device THERM 4 can be located on an outer surface of the second thermal block Thermal Block 2. The outer surface of the second thermal block Thermal Block 2 associated with the fourth temperature measuring device THERM 4 may be opposed to an outer surface of the second thermal block Thermal Block 2 associated with the third temperature measuring device THERM 3.

The temperature measuring devices THERM 1, THERM 2, THERM 3, THERM 4 are shown as blocks having height and width for illustrative purposes. The one or more of the temperature measuring devices THERM 1, THERM 2, THERM 3, THERM 4 may be a small device in relation to the thermal blocks Thermal Block 1, Thermal Block 2, and occupy a point or small area on or over the patient's skin or within the thermal blocks Thermal Block 1, Thermal Block 2.

The thermal blocks Thermal Block 1, Thermal Block 2 are shown as rectangles having height and width for illustrative purposes. One or more of the thermal blocks Thermal Block 1, Thermal Block 2 may be cubic, cylindrical, spherical, irregularly-shaped, and the like.

The first and second temperature measuring devices THERM 1, THERM 2 are separated by a distance m₁ of the first thermal block Thermal Block 1 and the third and fourth temperature measuring devices THERM 3, THERM 4 are separated by a distance m₂ of the second thermal block Thermal Block 2. In some embodiments, m₁ and m₂ can be substantially the same. In alternative embodiments, m₁ and m₂ can be different.

The first thermal block Thermal Block 1 can have a thermal conductivity k₁ and the second thermal block Thermal Block 2 can have a thermal conductivity k₂. Thermal conductivity can include the degree to which a specific material conducts heat. Thermal conductivity can be expressed in units of W/m° K. The first and second thermal blocks THERM 1 and THERM 2 can be the same material, such that k₁ and k₂ are substantially the same. In alternative embodiments, the first and second thermal blocks THERM 1 and THERM 2 can be different materials, such that k₁ and k₂ are different. One or more of the thermal blocks THERM 1, THERM 2 can comprise multiple materials such that k₁ and k₂ are not constant but are functions of the thicknesses of the multiple materials.

The first temperature measuring device THERM 1 can measure the temperature associated with the surface of the patient, such as at or near the patient's skin, at the first location, and the second temperature measuring device THERM 2 can measure the temperature through the first thermal block Thermal Block 1 at a distance m₁ from the first temperature measuring device THERM 1 at the first location.

The third temperature measuring device THERM 3 can measure the temperature associated with the surface of the patient, such as at or near the patient's skin, at the second location, and the fourth temperature measuring device THERM 4 can measure the temperature through the second thermal block Thermal Block 2 at a distance m₂ from the third temperature measuring device THERM 3 at the second location.

Examples of temperature measuring devices are, but not limited to, temperature sensors, resistance temperature detector, a thermocouple, semiconductor-based sensors, infrared sensors, bimetallic devices, thermometers, thermistors, change-of-state sensors, silicon diodes, and/or the like.

The temperature monitoring system of FIG. 1 illustrates two temperature monitoring devices. In some embodiments, the temperature monitoring system can include more than two temperature monitoring devices.

### Instrumentation-Sensors and Signal Processing Device

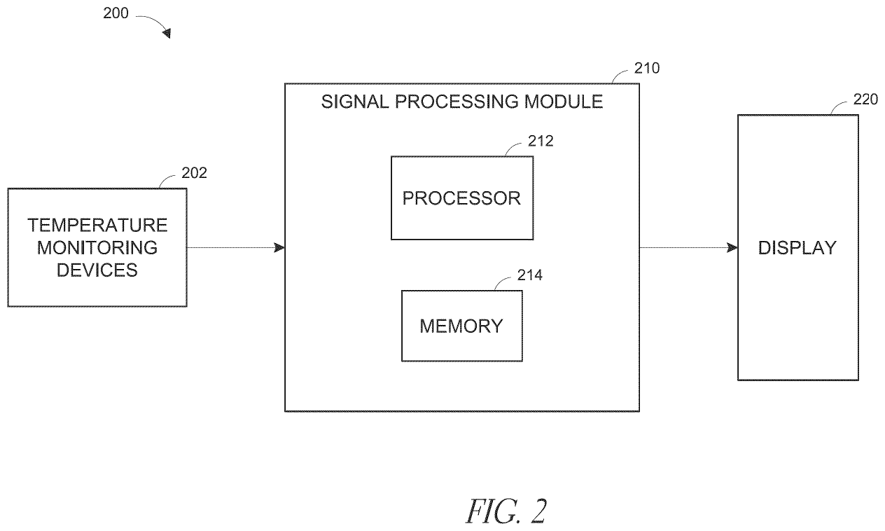

FIG. 2 is a block diagram of an example multi-site temperature monitoring system 200 according. The multi-site temperature monitoring system 200 can include one or more temperature monitoring devices 202, a signal processing module 210, and a display 220. The signal processing module 210 can include a processor 212 and a memory 214. The one or more temperature monitoring devices 202 each can include at least one temperature measuring device and a thermal block. The temperature monitoring devices 202, the signal processing module 210, and/or the display 220 can be connected via a cable or cables, wireless technology, Bluetooth®, and can communicate using near field communication protocols, Wi-Fi, and/or the like.

The temperature measurements from the temperature monitoring devices 202 can be received by the signal processing module 210 and stored in memory 214. The temperature monitoring devices 202 can transmit raw sensor data to the signal processing module 210, and the signal processing module 210 can convert the raw sensor data into data representing physiological parameters for transmission to the display 220. For example, temperature measurements can be analyzed by the processor 212 to estimate a patient's core body temperature. The processor 212 can transmit the estimated core body temperature to the display 220 to be displayed.

### Circuit Block Diagram Representation of Multi-Site Core Temperature Monitoring System

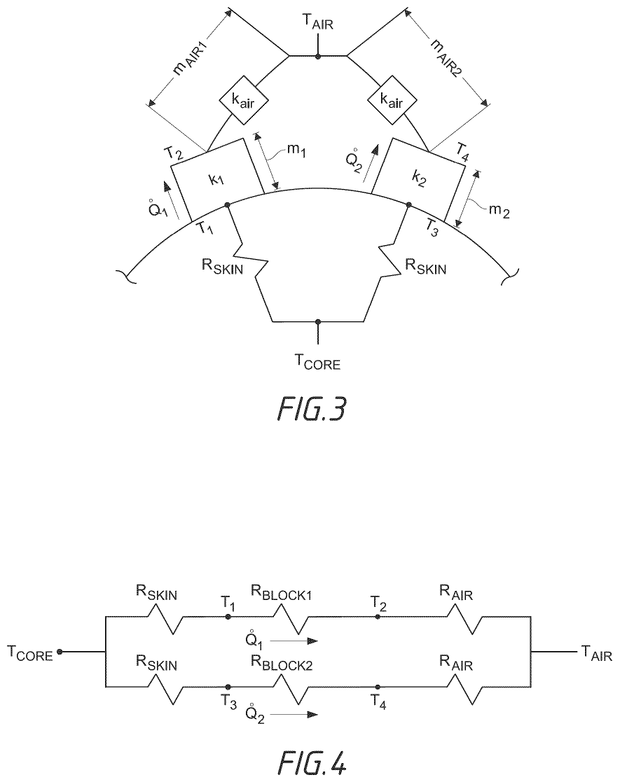

FIG. 3 is a circuit block diagram representation of an example multi-site temperature monitoring system. The temperature monitoring system can include a first temperature monitoring device that can include a first thermal block having a thermal conductivity k₁, a first temperature measuring device that can measure a first temperature T₁ and a second temperature measuring device that can measure a second temperature T₂. The temperature monitoring system can further include a second temperature measuring device that can include a second thermal block having a thermal conductivity k₂, a third temperature measuring device that can measure a third temperature T₃ and a fourth temperature measuring device that can measure a fourth temperature T₄.

The thermal conductivities k₁ and k₂ can be substantially the same. In alternative embodiments, the thermal conductivities k₁ and k₂ can be different. The two thermal blocks of FIG. 3 can include the first thermal block Thermal Block 1 and second thermal block Thermal Block 2 as illustrated in FIG. 1. The temperatures T₁, T₂, T₃, T₄ can be measured by the first, second, third, and fourth temperature measuring devices THERM 1, THERM 2, THERM 3, THERM 4, respectively, as illustrated in FIG. 1.

The distance m₁ is the distance between the first and second temperature measuring devices through the first thermal block and the distance m₂ is the distance between the third and fourth temperature measuring devices through the second thermal block. The distances m₁ and m₂ can be substantially the same. In alternative embodiments, the distances m₁ and m₂ can be different.

FIG. 3 also illustrates the thermal conductivity of air kₐᵢᵣ, the air temperature T_{AIR}, the patient's core body temperature T_{CORE}, and the thermal resistance of the patient's skin R_{SKIN}. Thermal resistance can be an indication of a material's resistance to heat flow. Thermal resistance can be expressed in units of ° K/W. FIG. 3 illustrates two distances, m_{AIR1} and m_{AIR2}, between a location on or within each thermal block, respectively, and a location in the air, indicated by T_{AIR}.

The temperature monitoring system of FIG. 3 illustrates heat transfer rates, Q̅₁ and Q̅₂, associated with the first and second thermal blocks, respectively. Heat transfer rates can include the rate at which the heat is transferred through a material. Heat transfer rates can be expressed in units of Watts. The heat transfer rates, Q̅₁ and Q̅₂, can indicate the heat transferred through the thermal blocks over a time interval.

The first temperature monitoring device and the second temperature monitoring device can be placed at different locations associated with the surface of the patient. For example, the first temperature monitoring device and the second temperature monitoring device can be placed at different locations on or near the patient's skin. The embodiment of the circuit block diagram in FIG. 3 illustrates two temperature monitoring devices. The multi-site core temperature monitoring system can include more than two temperature monitoring devices disposed on different locations associated with the surface of the patient. For example, the multi-site core temperature monitoring system can include three, four, five, more than five, six, etc. temperature monitoring devices disposed on different locations associated with the surface of the patient.

### Circuit Representation of Multi-Site Core Temperature Monitoring System

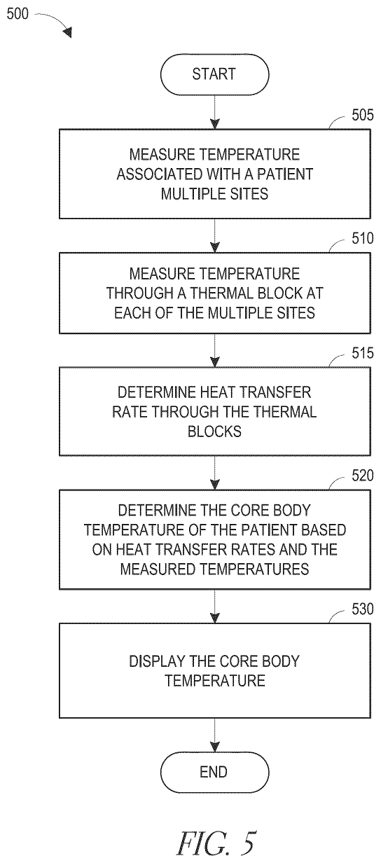

FIG. 4 is a circuit representation of the example multi-site core body temperature monitoring system illustrated in FIGS. 1 and 3. The circuit representation can include the patient's core body temperature T_{CORE}, the thermal resistance of the patient's skin R_{SKIN}, a thermal resistance of the first thermal block R_{BLOCK1}, a thermal resistance of the second thermal block R_{BLOCK2}, a thermal resistance of the air R_{AIR}, temperatures T₁, T₂, T₃, and T₄, the heat transfer rate Q̅₁ associated with the heat transfer of the first thermal block, the heat transfer rate Q̅₂ associated with the heat transfer of the second thermal block, and the air temperature T_{AIR}.

Equations based on the circuit representation can be used to determine the core body temperature T_{CORE} of the patient. The equations and calculations below illustrate one possible example that can be used to determine the core body temperature T_{CORE} of the patient using the measured temperatures T₁, T₂, T₃, and T₄, the known thermal conductivities k₁, k₂ of the first and second thermal blocks, respectively, and the distances m₁ and m₂ between the first and second temperature measuring devices and the third and fourth temperature measuring devices, respectively. In other embodiments, other calculations and equations can be used to determine the core body temperature T_{CORE} of the patient based on the temperature monitoring systems of FIGS. 1-4.

The thermal resistance R_{BLOCK1} and R_{BLOCK2} can be determined by:

$$\begin{matrix}{R}_{BLOCK 1} = \frac{1}{{k}_{1} {m}_{1}} \\ {R}_{BLOCK 2} = \frac{1}{{k}_{2} {m}_{2}}\end{matrix}$$

where:
k₁ is the thermal conductivity of the first thermal block Thermal Block 1;
k₂ is the thermal conductivity of the second thermal block Thermal Block 2;
m₁ is the distance through the first thermal block Thermal Block 1 between the first temperature measuring device THERM 1 and the second temperature measuring device THERM 2; and
m₂ is the distance through the second thermal block Thermal Block 2 between the third temperature measuring device THERM 3 and the fourth temperature measuring device THERM 4.

The heat transfer rates, Q̅₁ and Q̅₂, for each of the two thermal blocks can be determined by:

$$\begin{matrix}{\dot{Q}}_{1} = \frac{{T}_{2} - {T}_{1}}{{R}_{BLOCK 1}} \\ {\dot{Q}}_{2} = \frac{{T}_{4} - {T}_{3}}{{R}_{BLOCK 2}}\end{matrix}$$

where:
T₁ is the temperature measured by the first temperature measuring device THERM 1;
T₂ is the temperature measured by the second temperature measuring device THERM 2;
T₃ is the temperature measured by the third temperature measuring device THERM 3; and
T₄ is the temperature measured by the fourth temperature measuring device THERM 4.

The core body temperature of the patient T_{core} can be determined by:

$$\begin{matrix}{T}_{CORE} - {T}_{1} = {\dot{Q}}_{1} {R}_{SKIN} \\ {T}_{CORE} - {T}_{3} = {\dot{Q}}_{2} {R}_{SKIN}\end{matrix}$$

where R_{SKIN} represents the thermal resistance of the patient's skin.

With two equations for the core temperature and two unknowns (T_{CORE} and R_{SKIN}), each value can be determined. An example calculation is:

$$\begin{matrix}{T}_{CORE} = ( {\dot{Q}}_{2} {R}_{SKIN} ) + {T}_{3} \\ ( {\dot{Q}}_{2} {R}_{SKIN} ) + {T}_{3} - {T}_{1} = {\dot{Q}}_{1} {R}_{SKIN} \\ ( {\dot{Q}}_{2} {R}_{SKIN} ) - ( {\dot{Q}}_{1} {R}_{SKIN} ) = {T}_{1} - {T}_{3} \\ {R}_{SKIN} ( {\dot{Q}}_{2} - {\dot{Q}}_{1} ) = {T}_{1} - {T}_{3} \\ {R}_{SKIN} = \frac{{T}_{1} - {T}_{3}}{{\dot{Q}}_{2} - {\dot{Q}}_{1}} \\ {T}_{CORE} = {\dot{Q}}_{1} {R}_{SKIN} + {T}_{1} \\ {T}_{CORE} = {\dot{Q}}_{1} ( \frac{{T}_{1} - {T}_{3}}{{\dot{Q}}_{2} - {\dot{Q}}_{1}} ) + {T}_{1} \\ where , as shown above : \\ {\dot{Q}}_{1} = \frac{{T}_{2} - {T}_{1}}{{R}_{BLOCK 1}} ; \\ {R}_{BLOCK 1} = \frac{1}{{k}_{1} {m}_{1}} ; \\ {\dot{Q}}_{2} = \frac{{T}_{4} - {T}_{3}}{{R}_{BLOCK 2}} ; and \\ {R}_{BLOCK 2} = \frac{1}{{k}_{2} {m}_{2}} .\end{matrix}$$

Further, equations based on the circuit representation can be used to determine the temperature of the air T_{AIR}. In some embodiments, T_{AIR} represents the ambient air temperature. The equations and calculations below illustrate one possible example that can be used to solve for the temperature of the air T_{AIR} using the measured temperatures T₁, T₂, T₃, and T₄, the known thermal conductivities k₁, k₂ of the first and second thermal blocks, respectively, and the distances m₁ and m₂ between the first and second temperature measuring devices and the third and fourth temperature measuring devices, respectively. In other embodiments, other calculations and equations can be used to determine the air temperature T_{AIR} based on the temperature monitoring systems of FIGS. 1-4.

The temperature of the air T_{AIR} can be determined by:

$$\begin{matrix}{T}_{AIR} - {T}_{2} = {\dot{Q}}_{1} {R}_{AIR} \\ {T}_{AIR} - {T}_{4} = {\dot{Q}}_{2} {R}_{AIR}\end{matrix}$$

where:
R_{AIR} represents the thermal resistance of air;
T₂ is the temperature measured by the second temperature measuring device THERM 2;
T₄ is the temperature measured by the fourth temperature measuring device THERM 4;

$$\begin{matrix}{\dot{Q}}_{1} = \frac{{T}_{2} - {T}_{1}}{{R}_{BLOCK 1}} ; \\ {R}_{BLOCK 1} = \frac{1}{{k}_{1} {m}_{1}} ; \\ {\dot{Q}}_{2} = \frac{{T}_{4} - {T}_{3}}{{R}_{BLOCK 2}} ; and \\ {R}_{BLOCK 2} = \frac{1}{{k}_{2} {m}_{2}} .\end{matrix}$$

With two equations for the air temperature and two unknowns (T_{AIR} and R_{AIR}), each value can be determined. For example, the values of T_{AIR} and R_{AIR} can be determined in a similar manner as described above with respect to T_{CORE} and R_{SKIN}. In an aspect, the ambient temperature calculation can improve or refine the core body temperature calculation. The ambient temperature calculation can provide patient thermoregulation information. For example, the ambient temperature calculation may be useful in determining whether the patient is under thick insulation (i.e., a thick blanker), has little insulation (i.e., a shirt and no blanket), or has no insulation (patient's skin exposed to air).

### Flow Diagram for Multi-Site Core Temperature Monitoring System

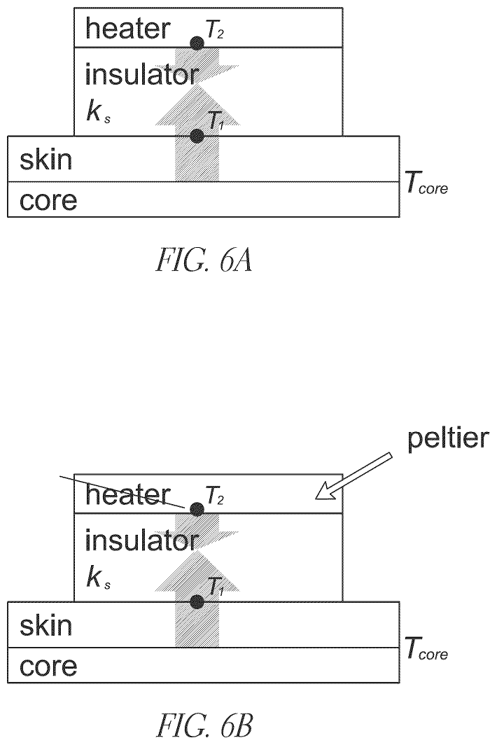

FIG. 5 is a flow diagram 500 illustrating an example process to accurately measure core body temperature using a multi-site temperature monitoring system. The flow diagram 500 can be implemented by the multi-site temperature monitoring system of FIGS. 1, 2, 3, and/or 4.

At block 505, the multi-site temperature monitoring system can measure temperature at multiple locations associated with the surface of the patient. For example, the first temperature measuring device THERM 1 of FIG. 1 can measure the temperature at or near the patient's skin at a first location, and the third temperature measuring device THERM 3 of FIG. 1 can measure the temperature at or near the patient's skin at a second location.

At block 510, the multi-site temperature monitoring system can measure temperature through thermal blocks at multiple sites. For example, the second temperature measuring device THERM 2 of FIG. 1 can measure the temperature through all or at least a portion of the first thermal block Thermal Block 1 at the first location, and the fourth temperature measuring device THERM 4 of FIG. 1 can measure the temperature through all or at least a portion of the second thermal block Thermal Block 2.

At block 515, the multi-site temperature monitoring system can determine the heat transfer rate through the thermal blocks at the multiple sites. For example, the processor 212 of FIG. 2 can receive the temperature measurements, and can use the heat transfer rate equations as described herein to determine the heat transfer rate through the thermal blocks based on the measured temperatures at block 505 and 510, the distance m₁ between the first and second temperature measuring devices THERM1, THERM 2, the distance m₂ between the third and fourth temperature measuring devices THERM 3, THERM 4, and the thermal conductivities k₁, k₂.

At block 520, the multi-site temperature monitoring system can determine thermal resistance of the patient's skin and/or the core body temperature of the patient based on the heat transfer rates and measured temperatures. For example, the processor 212 of FIG. 2 can use the core body temperature equations as described herein to determine the core body temperature T_{CORE} of the patient. In other aspects, other equations can be used.

At block 530, the multi-site temperature monitoring system can display the core body temperature of the patient. For example, the multi-site temperature monitoring system can transmit the core body temperature to the display 220 of FIG. 2.

### Heat Flux Measurement System

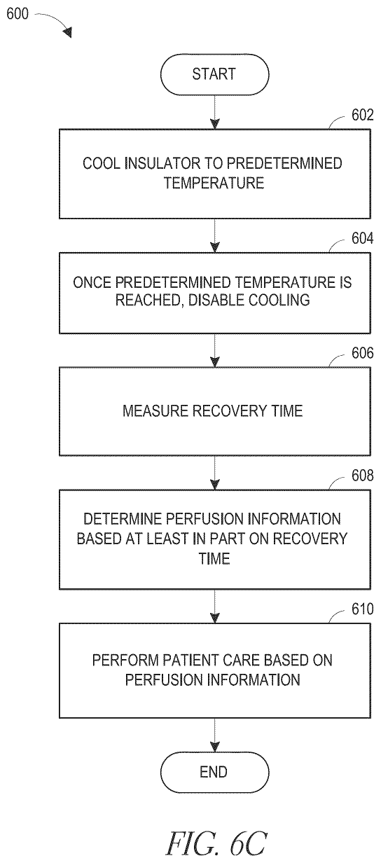

FIG. 6A is a block diagram of an example active heat flux measurement system according to certain embodiments. The active heat flux measurement system of FIG. 6A is like the passive heat flux measurement system of FIG. 1A, except the active heat flux measurement system of FIG. 6A can also include a heating component or heater over the insulator and thermistor T2. The heater can be cycled ON and OFF. For example, the active heat flux measurement system of FIG. 6A can further include a controller, such as a PID (proportional-integral-derivative) controller that operates a control loop feedback mechanism. The PID controller can control the heater operation and can repeatedly calculate the difference between the temperature measured at thermistor T1 and thermistor T2. The active heat flux measurement system of FIG. 6A can update the temperature difference measurement and can cycle the heater until thermistor T1 and thermistor T2 have approximately matching values. When the temperatures measured at T1 and T2 can be approximately the same, the net heat flux leaving the body can be approximately equal to the heat flux provided by the heater, and the measured temperature can approximate the core body temperature.

FIG. 6B is a block diagram of another example active heat flux measurement system. The active heat flux measurement system of FIG. 6B is like the active heat flux measurement system of FIG. 6A, except that the heater is replaced with a Peltier device. The Peltier effect can be an effect where a heat flux is created (i.e., heat is emitted or absorbed) when an electric current passes across the junction of two different types of materials. A Peltier device, such as a Peltier cooler, heater, or thermoelectric heat pump, can transfer heat from one side of the device to the other, with consumption of electrical energy, depending on the direction of the current. The difference between the active heat flux measurement system of FIG. 6A and the active heat flux measurement system of FIG. 6B, is that in the active heat flux measurement system of FIG. 6B, the Peltier device permits the controller to overheat or under heat the insulator, which impacts the heat flux.

For example, the active heat flux measurement system of FIG. 6B can overheat or under heat to a predetermined temperature and measure the recovery time. The recovery time can provide information about local skin perfusion, which can provide an indication of how the body is regulating temperature.

FIG. 6C is a flow chart of an example process 600 to obtain perfusion information of a patient using a Peltier active heat flux measurement system of FIG. 6B. At block 602, the Peltier device of the Peltier active heat flux measurement system can cool the insulator to a predetermined temperature. The predetermined temperature can be within a few degrees of the patient's skin temperature. The predetermined temperature can be approximately 1 degree less than the patient's skin temperature, approximately 2 degrees less than the patient's skin temperature, approximately 3 degrees less than the patient's skin temperature, approximately 5 degrees less than the patient's skin temperature, and the like. At block 604, the Peltier device can be turned OFF. At block 606, the recovery time between thermistor T1 and thermistor T2 can be measured. At block 608, perfusion information for the patient can be determined based at least in part on one or more of the recovery time, the temperature at thermistor T1 and the temperature at thermistor T2. At block, 610, the caregiver can perform patient care based on the perfusion information.

### Terminology

The embodiments disclosed herein are presented by way of examples only and not to limit the scope of the claims that follow. One of ordinary skill in the art will appreciate from the disclosure herein that many variations and modifications can be realized without departing from the scope of the present disclosure.

The term “and/or” herein has its broadest least limiting meaning which is the disclosure includes A alone, B alone, both A and B together, or A or B alternatively, but does not require both A and B or require one of A or one of B. As used herein, the phrase “at least one of “A, B, “and” C should be construed to mean a logical A or B or C, using a non-exclusive logical or.

The description herein is merely illustrative in nature and is in no way intended to limit the disclosure, its application, or uses. For purposes of clarity, the same reference numbers will be used in the drawings to identify similar elements. It should be understood that steps within a method may be executed in different order without altering the principles of the present disclosure.

As used herein, the term module may refer to, be part of, or include an Application Specific Integrated Circuit (ASIC); an electronic circuit; a combinational logic circuit; a field programmable gate array (FPGA); a processor (shared, dedicated, or group) that executes code; other suitable components that provide the described functionality; or a combination of some or all of the above, such as in a system-on-chip. The term module may include memory (shared, dedicated, or group) that stores code executed by the processor.

The term code, as used above, may include software, firmware, and/or microcode, and may refer to programs, routines, functions, classes, and/or objects. The term shared, as used above, means that some or all code from multiple modules may be executed using a single (shared) processor. In addition, some or all code from multiple modules may be stored by a single (shared) memory. The term group, as used above, means that some or all code from a single module may be executed using a group of processors. In addition, some or all code from a single module may be stored using a group of memories.

The apparatuses and methods described herein may be implemented by one or more computer programs executed by one or more processors. The computer programs include processor-executable instructions that are stored on a non-transitory tangible computer readable medium. The computer programs may also include stored data. Non-limiting examples of the non-transitory tangible computer readable medium are nonvolatile memory, magnetic storage, and optical storage. Although the foregoing invention has been described in terms of certain preferred embodiments, other embodiments will be apparent to those of ordinary skill in the art from the disclosure herein. Additionally, other combinations, omissions, substitutions and modifications will be apparent to the skilled artisan in view of the disclosure herein. Accordingly, the present invention is not intended to be limited by the reaction of the preferred embodiments, but is to be defined by reference to claims.

Additionally, all publications, patents, and patent applications mentioned in this specification are herein incorporated by reference to the same extent as if each individual publication, patent, or patent application was specifically and individually indicated to be incorporated by reference.

1-20. (canceled)

21. A core body temperature monitoring device, comprising:
a first temperature sensor configured to measure a first temperature associated with a first location of a user's skin;
a first material positioned between the first temperature sensor and the user's skin, the first material having a first thermal conductivity;
a second material positioned between the first temperature sensor and the user's skin, the second material having a second thermal conductivity different than the first thermal conductivity, wherein a total thermal conductivity of the first material and the second material is based on at least the first thermal conductivity and the second thermal conductivity, wherein the first temperature sensor is configured to measure the first temperature through the first material and the second material;
a second temperature sensor configured to measure a second temperature associated with a second location of the user's skin; and
a third material positioned between the second temperature sensor and the user's skin, wherein the second temperature sensor is configured to measure the second temperature through the third material,
wherein first and second measurements from the first and second temperature sensors are useable to estimate core body temperature of the user.

22. The core body temperature monitoring device of claim 21 further comprising a third temperature sensor associated with the first temperature sensor and configured to measure a third temperature associated with the first location of the user's skin, wherein measurements from the first, second, and third temperature sensors are useable to determine the core body temperature estimate of the user.

23. The core body temperature monitoring device of claim 22 further comprising a fourth temperature sensor associated with the second temperature sensor and configured to measure a fourth temperature associated with the second location of the user's skin, wherein measurements from the first, second, third, and fourth temperature sensors are useable to determine the core body temperature estimate of the user.

24. The core body temperature monitoring device of claim 21 further comprising a fourth material positioned between the second temperature sensor and the user's skin, wherein the second temperature sensor is configured to measure the second temperature through the third material and the fourth material.

25. The core body temperature monitoring device of claim 21, wherein the total thermal conductivity of the first material and the second material is different than a thermal conductivity of the third material.

26. The core body temperature monitoring device of claim 21 wherein the third material is the same as the first material.

27. The core body temperature monitoring device of claim 21 wherein the third material is different than the first material and the second material.

28. The core body temperature monitoring device of claim 21 wherein at least one of the first and second temperature sensors is a thermistor.

29. The core body temperature monitoring device of claim 21 wherein an ambient air temperature measurement is used to further refine the core body temperature estimate.

30. The core body temperature monitoring device of claim 21 further comprising a display configured to display the core body temperature estimate of the user.

31. A method of monitoring core body temperature of a user, comprising:
measuring a first temperature associated with a first location of the user's skin with a first temperature sensor through a first material and a second material, the first material having a first thermal conductivity, the second material having a second thermal conductivity different than the first thermal conductivity, wherein a total thermal conductivity of the first material and the second material is based on at least the first thermal conductivity and the second thermal conductivity;
measuring a second temperature associated with a second location of the user's skin with a second temperature sensor through a third material; and
estimating the core body temperature of the user from the first and second measurements from the first and second temperature sensors.

32. The method of claim 31 further comprising:
measuring a third temperature associated with the first location of the user's skin with a third temperature sensor associated with the first temperature sensor; and
estimating the core body temperature of the user from the first, second, and third measurements from the first, second, and third temperature sensors.

33. The method of claim 32 further comprising:
measuring a fourth temperature associated with the second location of the user's skin with a fourth temperature sensor associated with the second temperature sensor; and
estimating the core body temperature of the user from the first, second, third, and fourth measurements from the first, second, third, and fourth temperature sensors.

34. The method of claim 31 comprising measuring the second temperature associated with the second location of the user's skin with the second temperature sensor through the third material and a fourth material.

35. The method of claim 31 wherein the total thermal conductivity of the first material and the second material is different than a thermal conductivity of the third material.

36. The method of claim 31 wherein the third material is the same as the first material.

37. The method of claim 31 wherein the third material is different than the first material and the second material.

38. The method of claim 31 wherein at least one of the first and second temperature sensors is a thermistor.

39. The method of claim 31 further comprising refining the estimated core body temperature with an ambient air temperature measurement.

40. The method of claim 31 further comprising displaying the estimated core body temperature of the user.
